(19) United States
(12) Patent Application Publication
(10) Pub. No.: US 2025/0375168 A1
(43) Pub. Date: Dec. 11, 2025

(54) IMAGE PROCESSING DEVICE, IMAGE PROCESSING METHOD, AND STORAGE MEDIUM

(71) Applicant: NEC Corporation, Tokyo (JP)

(72) Inventor: Kazuhiro WATANABE, Tokyo (JP)

(73) Assignee: NEC Corporation, Tokyo (JP)

(21) Appl. No.: 19/220,317

(22) Filed: May 28, 2025

(30) Foreign Application Priority Data
Jun. 7, 2024 (JP) 2024-092885

(51) Int. Cl. A61B 5/00

(52) U.S. Cl. CPC A61B 5/7267; A61B 5/743; A61B 5/7435; A61B 5/748

(57) ABSTRACT

The image processing device 1X includes an image acquisition means 30X, an option generation means 33X, and a display control means 34X. The image acquisition means 30X acquires a medical image. The option generation means 33X generates, based on the medical image, plural options of a size of a region of interest included in the medical image. The display control means 34X causes a display device to display information on the plural options of the size of the region of interest. With the displayed information, healthcare worker's decision making is assisted, for example.

### INCORPORATION BY REFERENCE

This application is based upon and claims the benefit of priority from Japanese Patent Application No. 2024-092885, filed on Jun. 7, 2024, the disclosure of which is incorporated herein in its entirety by reference.

### TECHNICAL FIELD

The present disclosure relates to a technical field of an image processing device, an image processing method, and a program using a medical image.

### BACKGROUND

An image processing system for detecting a lesion from a medical image of a patient is known. For example, Patent Literature 1 discloses a medical image processing device for identifying the lesion region from the medical image obtained by photographing a lung to estimate whether the lung node of the identified lesion region is a benign or a malignancy.

### CITATION LIST

### Patent Literature

Patent Literature 1: JP 2016-007270A

### SUMMARY

In general, in estimating the size of a region of interest, such as a lesion region, in a medical image, it is performed to infer the coordinates of the region of interest using a regression model. In this case, an inference result indicating a single proposal per region of interest is obtained, and therefore it cannot present to the user alternatives even if the inference result is clearly wrong.

In view of the above-described issues, one object of the present disclosure is to provide an image processing device, an image processing method, and a storage medium capable of presenting information on a region of interest in consideration of the possibility of an error in the inference.

In an example aspect of the present disclosure, there is provided an image processing device including:
an image acquisition means configured to acquire a medical image;
an option generation means configured to generate, based on the medical image, plural options of a size of a region of interest included in the medical image; and
a display control means configured to cause a display device to display information on the plural options.

In an example aspect of the present disclosure, there is provided an image processing method executed by a computer, including:
acquiring a medical image;
generating, based on the medical image, plural options of a size of a region of interest included in the medical image; and
causing a display device to display information on the plural options.

In an example aspect of the present disclosure, there is provided a program executed by a computer, the program causing the computer to:
acquire a medical image;
generate, based on the medical image, plural options of a size of a region of interest included in the medical image; and
cause a display device to display information on the plural options.

An example advantage according to the present disclosure is present information regarding a region of interest in consideration of the possibility of error in inference.

### BRIEF DESCRIPTION OF THE DRAWINGS

FIG. 1 illustrates the outline configuration of a lesion evaluation system.

FIG. 2 illustrates an outline processing performed by an image processing device.

FIG. 3 illustrates an example of a functional block of a processor of the image processing device.

FIG. 4 illustrates a histogram of lesion size options.

FIG. 5 illustrates a first display example of information on lesion size options.

FIG. 6 illustrates a second display example of information on lesion size options.

FIG. 7 illustrates an example of a flowchart showing an outline of the processing performed by the image processing device.

FIG. 8 illustrates the outline configuration of the lesion evaluation system.

FIG. 9 illustrates a block diagram of an image processing device.

FIG. 10 illustrates an example of a flowchart showing a procedure of the process performed by the image processing device.

### EXAMPLE EMBODIMENTS

Hereinafter, example embodiments of an image processing device, an image processing method, and a program will be described with reference to the drawings.

### First Example Embodiment

### (1) System Configuration

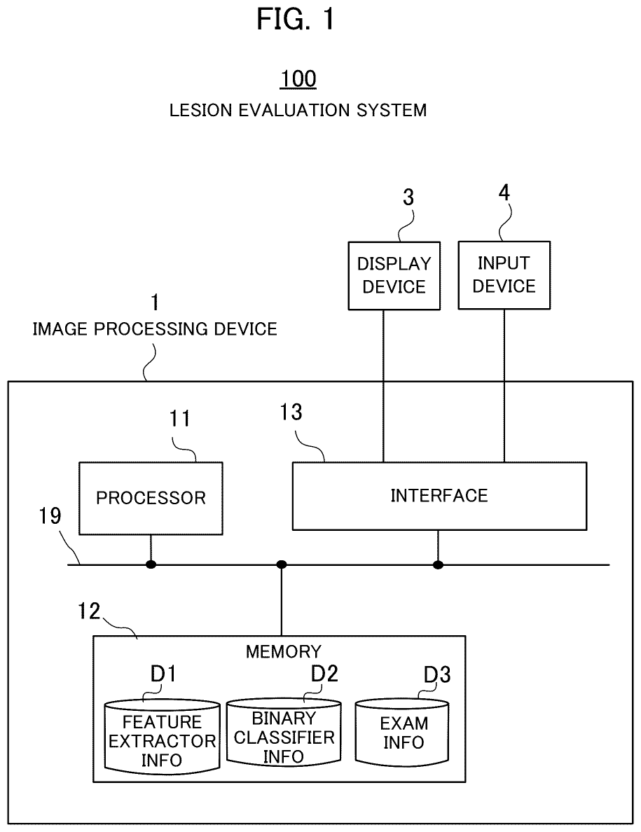

FIG. 1 shows a schematic configuration of a lesion evaluation system 100. The lesion evaluation system 100 shown in FIG. 1 is a system which evaluates the lesion (condition) of an examinee such as a patient and presents the evaluation result to the medical worker such as a doctor to thereby support healthcare (medical) worker's decision making (including diagnosis support and trial support). The lesion evaluation system 100 mainly includes an image processing device 1, a display device 3, and an input device 4.

The image processing device 1 estimates the lesion size on the basis of the medical image obtained through the examination of the examinee and presents information on the estimated lesion size to the user. In this case, the image processing device 1 performs display control of the display device 3 or performs various processing based on the user input signal received from the input device 4.

The term “medical image” herein indicates an image acquired through the examination of an organ of the examinee. Examples of medical images include CT images obtained by CT examination, MRI images obtained by MRI examination, endoscopic images obtained by endoscopic examination, images obtained by X-ray examination, images obtained by echography, and images obtained by any other examination. Also, the term “lesion size” herein indicates the size of the lesion region which appears in the medical image, and examples of the lesion size include the long diameter of the lesion region and the length of the diagonal of the rectangle when the lesion region is regarded as a rectangle. It is noted that the lesion size may be the actual size of the lesion to be estimated by any method from the medical image, or may be the size on the medical image of the lesion. The lesion size is an example of “size of region of interest”. The term “region of interest” does not necessarily refer to a lesion region and may refer to any region on the medical image to be detected in the examination. However, as a representative example, the region of interest is assumed to be a lesion region in the following example embodiment.

The display device 3 performs a predetermined display based on the display signal supplied from the image processing device 1. Examples of the display 3 include a display, such as a CRT (Cathode Ray Tube) and a LDC (Liquid Crystal Display), and a projector.

The input device 4 generates a user input signal based on an operation by a user of the image processing device 1 such as a doctor. Examples of the input device 4 include buttons, a keyboard, a pointing device such as a mouse, a touch panel, a remote controller, a voice input device, and any other user interface.

Further, in FIG. 1, an example of a hardware configuration of the image processing device 1 is shown. The image processing device 1 mainly includes a processor 11, a memory 12, and an interface 13. These elements are connected to one another via a data bus 19.

The processor 11 executes a predetermined process by executing a program or the like stored in the memory 12. The processor 11 is a processor such as a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by various volatile memories used as working memories such as a RAM (Random Access Memory) and a ROM (Read Only Memory) and non-volatile memories for storing the information necessary for the image processing device 1 to process data. The memory 12 may include an external storage device, such as a hard disk, that is connected to or embedded in the image processing device 1, or may include a storage medium, such as a removable flash memory. The memory 12 stores programs and other information necessary for the image processing device 1 to execute the processing according to the present example embodiment.

The memory 12 store feature extractor information D1, binary classifier information D2, and examination information D3.

The feature extractor information D1 is information regarding a feature extractor for converting a medical image into a feature vector in a predetermined number of dimensions and includes parameters for configuring the feature extractor. The feature extractor may be any model that performs feature extraction from an image. For example, the feature extractor is a machine learning model based on deep learning and the like, and the learned parameters are stored in advance as the feature extractor information D1. If the feature extractor is a neural-network-based model, the feature extractor information D1 includes parameters such as layer structure, neuron structure of each layer, number of filters and filter size in each layer, and weight for each element of each filter.

The binary classifier information D2 is information regarding “N” binary classifiers and includes parameters for constructing respective N binary classifiers. The number Nis the number of bits required to represent the coordinates for identifying the lesion region in binary, and binary classifiers are machine learning models configured to infer the value (0 or 1) of the respective bit positions of the binary. Each binary classifier takes, as an input, the feature vector output by the feature extractor or the data based on the feature vector and outputs, as an inference result, a score representing the probability of being 1in the binary classification problem of 0 or 1. Thus, each binary classifier is a model trained, through machine learning, to learn a relation between a feature vector of a medical image and a value for the associated bit position of a region of interest (here, a lesion region) existing in the medical image. Then, the parameters of the learned binary classifiers are stored in advance as the binary classifier information D2.

The examination information D3 is the examination information obtained through the examination of the examinee, and at least includes a medical image group of the examinee.

The interface 13 performs interface operation between the image processing device 1 and external devices. For example, the interface 13 is electrically connected to the display device 3 and the input device 4. The interface 13 may be a communication interface such as a network adapter for wired or wireless communication with an external device, or may be a hardware interface compliant with a USB (Universal Serial Bus), SATA (Serial AT Attachment), and the like. The interface 13 may perform an interface operation with an external device such as the display device 3 and the input device 4 via a communication network such as the Internet.

The configuration of the lesion evaluation system 100 shown in FIG. 1 is an example, and various change may be performed thereto.

For example, the image processing device 1 may be configured integrally with at least either the display device 3 and/or the input device 4. In another example, the image processing device 1 may include a sound output device that outputs information by sound. In yet another example, the image processing device 1 may be configured by a plurality of devices. In another example, the image processing device 1 may receive the medical image from an image generator that generates a medical image of the examinee instead of previously storing the examination information D3 in the memory 12.

### (2) Process Overview

First, an outline of the processing that is executed by the image processing device 1 in the present embodiment will be described. The image processing device 1 uses the feature vector generated from the medical image of the examinee to infer the values of the respective bit positions representing the coordinates of the lesion region by using the binary classifiers, and compares the inference results with a plurality of sets of threshold value(s) to generate a plurality of options of the lesion size.

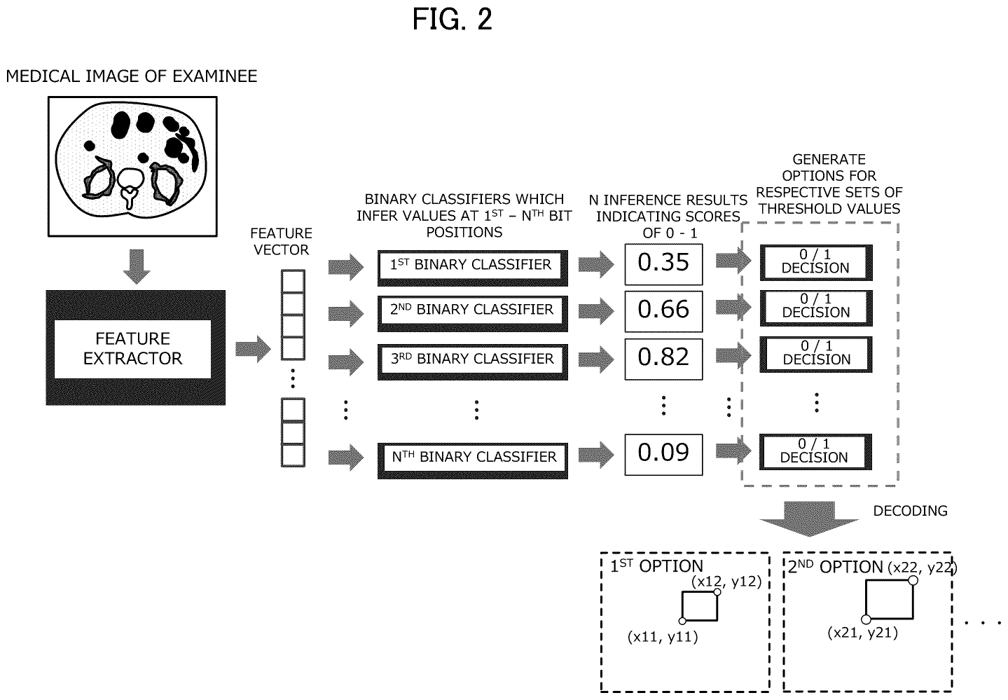

FIG. 2 is a diagram illustrating an outline of processing that is executed by the image processing device 1. In FIG. 2, first, the image processing device 1 inputs the medical image of the examinee to the feature extractor and obtains the feature vector of the input medical image from the feature extractor. Then, the image processing device 1 inputs the feature vector to N binary classifiers (the first binary classifier to the N-th binary classifier) which infer the values (0 or 1) of respective bit positions representing the coordinates of the lesion region. The N binary classifiers output inference results (i.e., scores ranging from 0 to 1) indicating the probability that the value of the corresponding bit position will be 1.

In this example embodiment, the coordinates of the lesion region represented by binary are assumed to be the coordinates of the two diagonal points of the lesion region provided that the lesion region is a rectangular. In this case, provided that the X-coordinate is expressed by 8 bits and the y-coordinate is expressed by 8 bits in the two-dimensional coordinate (x-y coordinate) system on the medical image, the number N is 32 (=8×2×2). In this case, the 32 binary classifiers output the probabilities that the value (0 or 1) corresponding to each bit position of the 32 bits representing the coordinates of the two diagonal points is classified into 1.

Then, the image processing device 1 compares the inference result from each binary classifier with a threshold value, and makes a determination (“0/1 decision” in the figure) as to whether each bit position indicating the coordinates of the diagonal points specifying the lesion region is either 1 or 0. The threshold values used herein may be set to different values for respective bit positions or may be set to a unified value in common for all bit positions.

Then, the image processing device 1 decodes the binary values according to the obtained threshold determination results for respective bit positions, thereby identifying the coordinates of the diagonal points of the lesion region in decimal notation. Thus, the image processing device 1 recognizes an option (alternative) of the lesion region on the medical image. The image processing device 1 acquires the threshold determination results for respective bit positions using another set of the threshold value(s) different from the set of the threshold value(s) already used, and thereby identifies the coordinates of the diagonal points of the lesion region based on the threshold determination results. Thus, the image processing device 1 recognizes a new option (another alternative) of the lesion region on the medical image. In FIG. 2, at least a first option that is a rectangular lesion region having (x11, y11) and (x12, y12) as diagonal points, and a second option that is a rectangular lesion region having (x21, y21) and (x22, y22) as diagonal points are obtained. Thus, the image processing device 1 generates a plurality of options of the pair of diagonal points by using different sets of the threshold value(s).

The coding method for expressing the coordinates of two diagonal points in binary notation is not limited to the method for converting decimal to binary, and any coding method may be used. For example, the coordinates of two diagonal points may be coded using any one of the alpha sign, Johnson sign, B1JDJn sign, B1JDJ sign, B2JDJ sign, or HEXJ sign. As for the above-described feature extractor, binary classifier, and encoding method, for example, specific examples are disclosed in the following literature.

Deval Shah, Zi Yu Xue, Tor M. Aamodt, Label Encoding for Regression Networks, arXiv:2212.01927.

Hereinafter, an example where the lesion region is identified by two diagonal points of the rectangle will be described as a representative example. However, the lesion region may not be rectangular and may be identified by three or more points.

Then, the image processing device 1 calculates the lesion sizes of acquired respective options of the lesion region and presents information on the lesion sizes corresponding to respective options to the examiner. Thus, the image processing device 1 can present information regarding a plurality of possible lesion sizes to the examiner in consideration of the possibility of errors in the inference result.

### (3) Functional Block

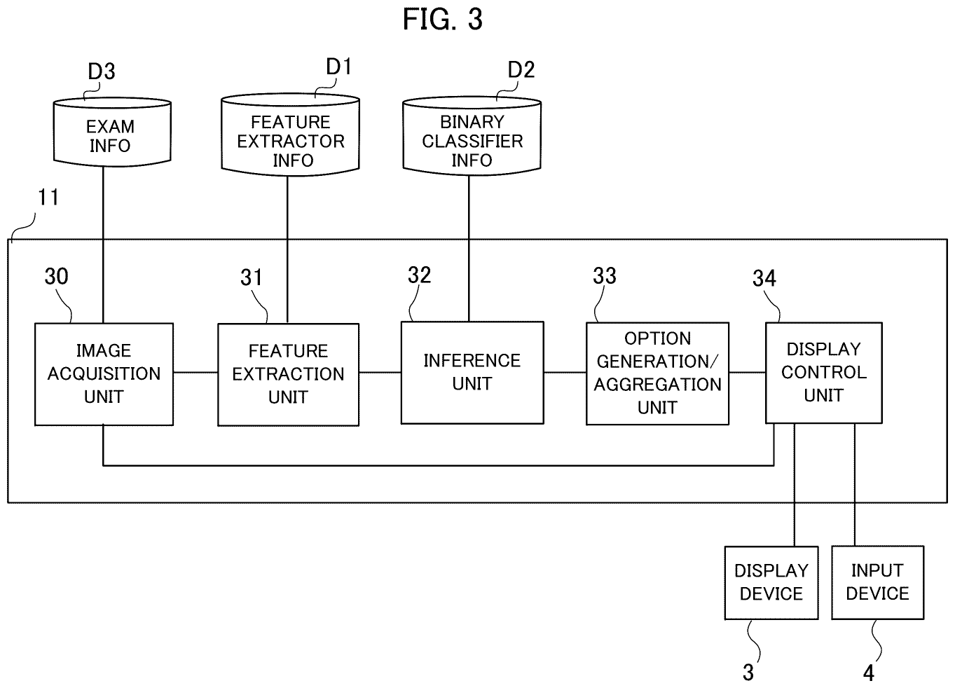

FIG. 3 is an example of a functional block of the processor 11 of the image processing device 1. The processor 11 of the image processing device 1 functionally includes an image acquisition unit 30, a feature extraction unit 31, an inference unit 32, an option generation/aggregation unit 33, and a display control unit 34. In FIG. 3, blocks to exchange data with each other are connected by a solid line, but the combination of blocks for exchanging data is not limited thereto. The same applies to the drawings of other functional blocks described below.

The image acquisition unit 30 acquires the medical image of the examinee included in the examination information D3 through the interface 13. Then, the image acquisition unit 30 supplies the acquired medical image to the feature extraction unit 31 and the display control unit 34, respectively. For example, the image acquisition unit 30 specifies the target medical image of evaluation in response to the user input signal received from the input device 4, and acquires the specified medical image.

The feature extraction unit 31 acquires the feature vector of the medical image from the feature extractor by inputting the medical image acquired by the image acquiring unit 30 to the feature extractor configured by referring to the feature extractor information D1. Then, the feature extraction unit 31 supplies the acquired feature vector to the inference unit 32. It is noted that the feature extractor may output features (feature data) in any tensor format which is not necessarily a vector format.

Based on the feature vector extracted by the feature extraction unit 31 and the N binary classifiers configured by referring to the binary classifier information D2, the inference unit 32 acquires the inference results on the binary classification at the N bit positions representing the coordinates of the diagonal points of the lesion region. In this case, for example, the inference unit 32 inputs the feature vector to the respective binary classifiers and then obtains scores output from the respective binary classifiers. The score represents the probability that the bit position corresponding to the binary classifier which outputs the score is 1. The inference unit 32 supplies the scores of the N bit positions to the option generation/aggregation unit 33.

The option generation/aggregation unit 33 compares the scores at the N bit positions with the threshold value(s) to determine whether each of the N bit positions is either 0 or 1. Then, the option generation/aggregation unit 33 decodes the N-digit binary number obtained through the determination into a decimal number, and converts the decimal number into coordinates of two diagonal points representing the lesion region. Then, the option generation/aggregation unit 33 acquires the coordinates of “M” (M is an integer of 2 or more) pairs of the diagonal points corresponding to the M sets of the threshold value(s) that are determined in advance or by stochastic sampling, thereby identifying M options of the lesion size. A set of threshold value(s) may be set to different values for respective bit position, or may be set a unified value regardless of the bit positions.

Further, the option generation/aggregation unit 33 generates a frequency distribution by aggregating the M options of the lesion size. With this frequency distribution, the image processing device 1 can grasp the most reliable lesion size or suitably grasp the worst lesion size (i.e., the largest lesion size) or the like. The option generation/aggregation unit 33 supplies the coordinates of the diagonal points and the corresponding lesion size, and the frequency distribution to the display control unit 34.

The display control unit 34 generates display information based on the medical image supplied from the image acquisition unit 30, the coordinates of the diagonal points supplied from the option generation/aggregation unit 33 and the corresponding lesion size, and the frequency distribution. Then, the display control unit 34 supplies the generated display information to the display device 3 to display information on a plurality of options of the lesion size on the display device 3. The display example will be described later.

Each component of the image acquisition unit 30, the feature extraction unit 31, the inference unit 32, the option generation/aggregation unit 33, and the display control unit 34 can be realized, for example, by the processor 11 executing a program. The necessary programs may be recorded on any non-volatile storage medium and installed as necessary to realize each component. It should be noted that at least a part of these components may be implemented by any combination of hardware, firmware, and software, or the like, without being limited to being implemented by software based on a program. At least some of these components may also be implemented using a user programmable integrated circuit such as a FPGA (Field-Programmable Gate Array) and a microcontroller. In this case, the integrated circuit may be used to realize a program to function as each of the above components. Further, at least some of the components may be realized by ASSP (Application Specific Standard Produce), ASIC (Application Specific Integrated Circuit), or quantum processor (quantum computer control chip). Thus, each component may be implemented by various hardware. The above is also true for other example embodiments described later. Furthermore, each of these components may be implemented by the cooperation of a plurality of computers, for example, using cloud computing technology.

### (4) Generation of Frequency Distribution

Next, the generation of the frequency distribution by the option generation/aggregation unit 33 will be described.

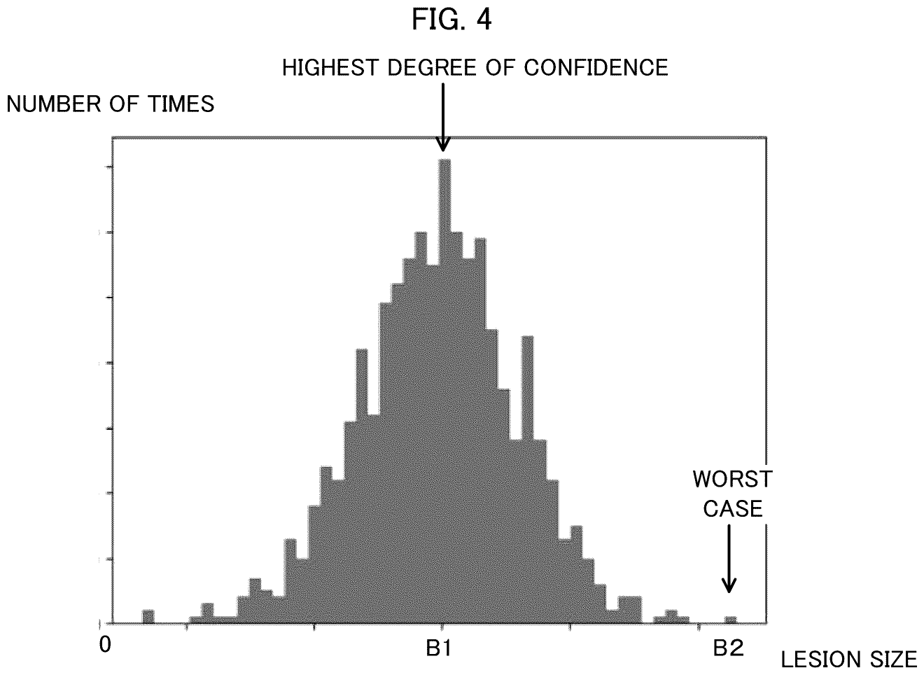

FIG. 4 shows a histogram of M options of the lesion size that are calculated by the option generation/aggregation unit 33. In FIG. 4, bins (classes) each having a predetermined bin width are set over a predetermined value range of possible lesion sizes, and M options of the lesion size are classified into corresponding bins. FIG. 4 illustrates that the bin B2 is the highest frequency (largest number of times) and the bin B1 indicates the largest size among the bins where the number of times is one or more.

In this instance, for example, the option generation/aggregation unit 33 deems the size corresponding to the bin B1 to be the lesion size with the highest degree of confidence. In addition, the option generation/aggregation unit 33 identifies the size corresponding to the bin B2 as the worst-case lesion size. In this way, the option generation/aggregation unit 33 can statistically grasp the possible lesion size with the highest degree of confidence and the worst-case lesion size, based on the histogram of M options of the lesion size. In some embodiments, the option generation/aggregation unit 33 may identify the worst-case lesion size among the bins where the number of times is equal to or larger than a predetermined number, which is more than 1.

Here, a supplementary description will be given of the setting of M sets of the threshold value(s) for generating M options of the lesion size.

The option generation/aggregation unit 33 generates M sets of the N threshold values to be compared with the inference results corresponding to the N bit positions. The N threshold values may be set to different values among all bit positions, or may be set to a unified value regardless of the bit positions.

In the first example, at least M sets of N threshold values corresponding to respective bit positions are stored in the memory 12 or the like, the option generation/aggregation unit 33 repeats setting of the N threshold values M times by referring to the memory 12. In the second example, the option generation/aggregation unit 33 determines N threshold values by probabilistic sampling (random extraction) for each of the M sets. In this case, an appropriate value range of the threshold values is determined in advance by experimental trials or the like, and the option generation/aggregation unit 33 sets the threshold values probabilistically within the value range. Here, if different threshold values are set depending on the bit positions, the above-described value ranges may be predetermined for respective threshold values depending on the bit positions through experimental trials or the like. The information on the above-described value ranges is previously stored in the memory 12 or the like.

### (5) Display Examples

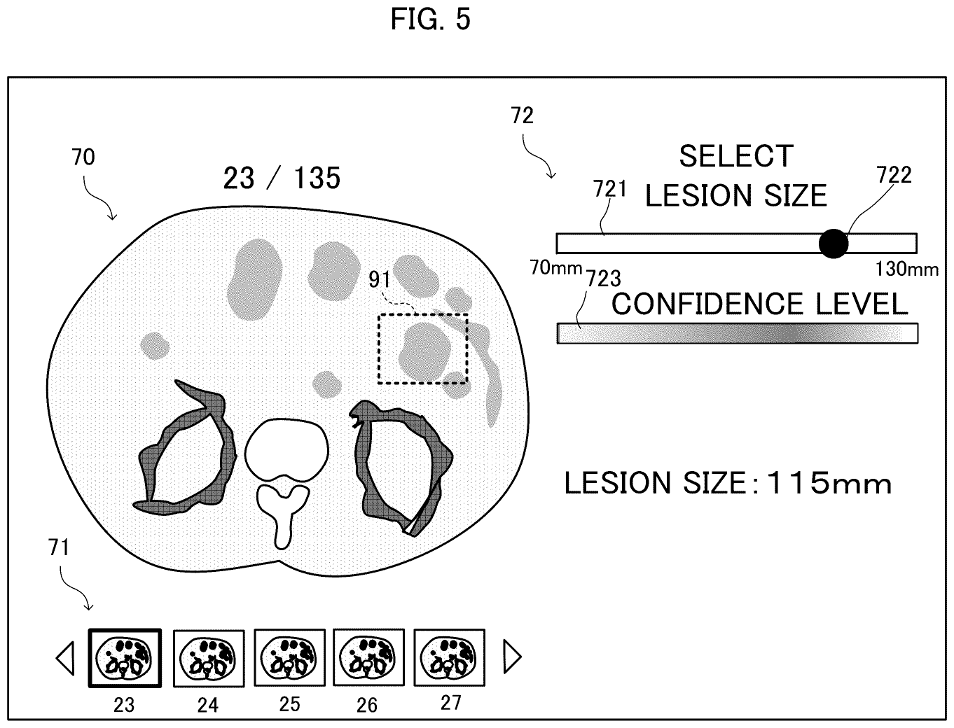

FIG. 5 shows a first display example of information on options for the lesion size. The display control unit 34 generates the display information based on the information supplied from the image acquisition unit 30 and the option generation/aggregation unit 33, and then transmits the generated display information to the display device 3 to display the display screen shown in FIG. 5 on the display device 3. In the display example shown in FIG. 5, the display control unit 34 of the image processing device 1 provides an image display area 70, an image selection area 71, and a lesion size related area 72 on the display screen.

The display control unit 34 displays on the image display area 70 the target medical image of the evaluation that is selected based on the user operation to the input device 4 from an examination image group. In the example shown in FIG. 5, the display control unit 34 displays the 23rd medical image of the medical image group of the examinee that contains 135 sheets. The display control unit 34 displays over the image a bounding box 91 indicating an option of the lesion region having the selected lesion size in the lesion size related area 72.

Further, the display control unit 34 displays on the image selection area 71 a part of the examination image group similar to the image displayed on the image display area 70 as thumbnail images. The display control unit 34 may display all of the thumbnail images of the examination image group on the image selection area 71. Upon detecting a predetermined operation, such as scroll operation through the input device 4, other than the selection operation on the image selection area 71, the display control unit 34 may also switch the image displayed on the image display area 70 accordingly.

The display control unit 34 displays information on options of the lesion size identified from the target medical image of the evaluation displayed on the image display area 70 in the lesion size related area 72. On the lesion size related area 72, the lesion size bar 721 and a confidence bar 723 are provided.

The lesion size bar 721 is a bar representing a value range of the options of the lesion size identified from medical images, and a slider 722 is provided for selecting a specific lesion size from the value range of the option of the lesion size. Then, the display control unit 34 displays on the medical image of the image display area 70 a bounding box 91 indicating the option of the lesion region corresponding to the lesion size (here, 115 mm) specified by the slider 722. Thus, the display control unit 34 highlights the option of the lesion region corresponding to a specific size selected from the options of the lesion size by the bounding box 91 on the target medical image of evaluation. If there are a plurality of options of the lesion region corresponding to the lesion size specified by the slider 722, the display control unit 34 will display a bounding box 91 indicating an option of the lesion region selected randomly or based on a predetermined rule. Further, the display control unit 34 displays the numerical value (115 mm in this case) of the lesion size specified by the slider 722 on the lesion size related area 72.

The confidence bar 723 has the same length as the lesion size bar 721 and represents the degrees of confidence of the options of the lesion size at respective positions on the lesion size bar 721 as a heat map. Here, the darker the color of the position corresponding to an option of the lesion size is, the higher the degree of confidence of the option becomes. The degree of confidence herein increases with an increase in the number of times on the corresponding bin in the histogram (frequency distribution) shown in FIG. 4. In some embodiments, the degree of confidence is normalized so that the degree of confidence of the option (lesion size classified in the bin B1 in FIG. 4) having the highest frequency is a predetermined value (e.g., 1) and the degree of confidence of the option having zero frequency is 0. Thus, by providing the confidence bar 723, the display control unit 34 can enable the examiner to visually grasp the degree of confidence of the option of the lesion size. The display control unit 34 may display the histogram shown in FIG. 4 in association with the lesion size bar 721 instead of the confidence bar 723.

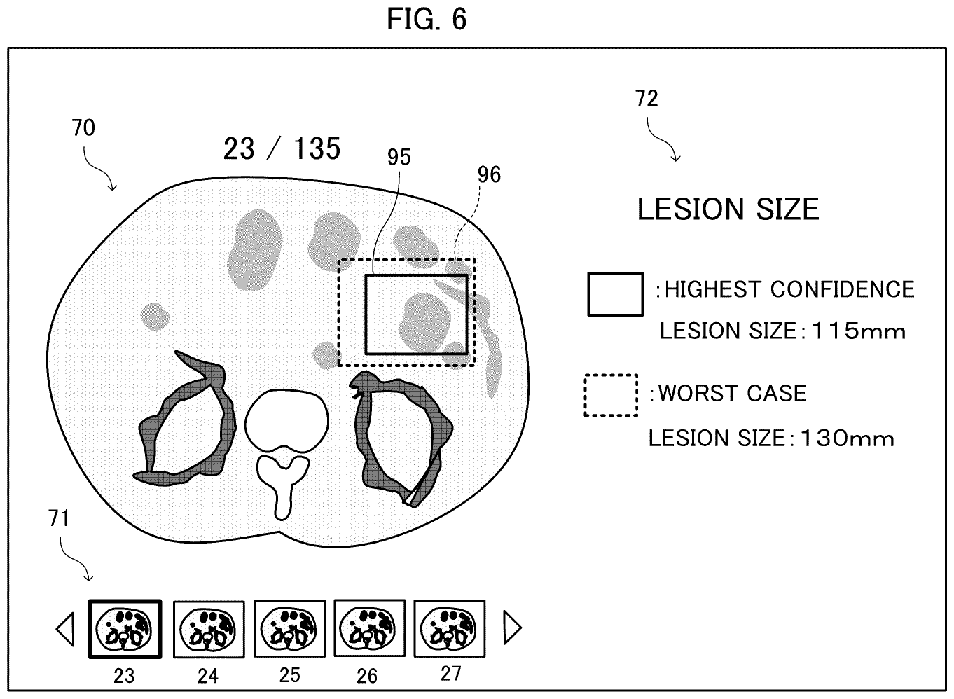

FIG. 6 shows a second display example of information on the options of the lesion size. The display control unit 34 of the image processing device 1 provides an image display area 70, an image selection area 71, and a lesion size related area 72, on the display screen.

In the second display example, the display control unit 34 displays the medical image on the image display area 70 while superimposing the bounding box 95 indicating the option of the lesion region having the highest degree of confidence and the bounding box 96 indicating the worst-case option of the lesion region. Further, the display control unit 34 displays, on the lesion size related area 72, the line style (solid line in this case) of the bounding box 95 of the option of lesion region having the highest degree of confidence degree and the corresponding lesion size (here 115 mm). In the same way, the display control unit 34 displays the line style (here dashed line) of the bounding box 96 of the worst-case option of the lesion region and the corresponding lesion size (130 mm here).

For example, upon receiving the frequency distribution shown in FIG. 4 is supplied from the option generation/aggregation unit 33, the display control unit 34 determines that the lesion size indicated by the bin B1 has the highest degree of confidence and therefore displays the bounding box 95 indicating the option of the lesion region classified into the bin B1. Since there are a plurality of options of the lesion regions classified into the bin B1, the display control unit 34 may, for example, select one option of the lesion region randomly or based on a predetermined rule from these options, and display the bounding box 95 indicating selected option of the lesion region. Further, the display control unit 34 identifies the option of the lesion region classified into bin B2 representing the largest lesion size among the options of the lesion size, and displays the bounding box 96 indicating the identified option of the lesion region.

Thus, in the second display example, the image processing device 1 presents a plurality of options of the lesion region and the lesion size for a specific lesion to the examiner simultaneously. Thus, the image processing device 1 can visualize a plurality of chances of the lesion.

The display examples shown in FIG. 5 and FIG. 6 are just examples, various changes may be applied thereto. For example, the display control unit 34 may determine a display mode (e.g., a color, a line style, or/and a thickness of a line, and the like) of each bounding box based on the degree of confidence of the lesion size corresponding to each bounding box. In another example, the display control unit 34 may simultaneously display three or more options of the lesion region in the second display example. In still other instances, the display control 34 may further display RECIST (Response Evaluation Criteria in Solid Tumors) evaluation, the drug effect evaluation, or the outcome of a treatment effectiveness determination of the patient.

### (6) Processing Flow

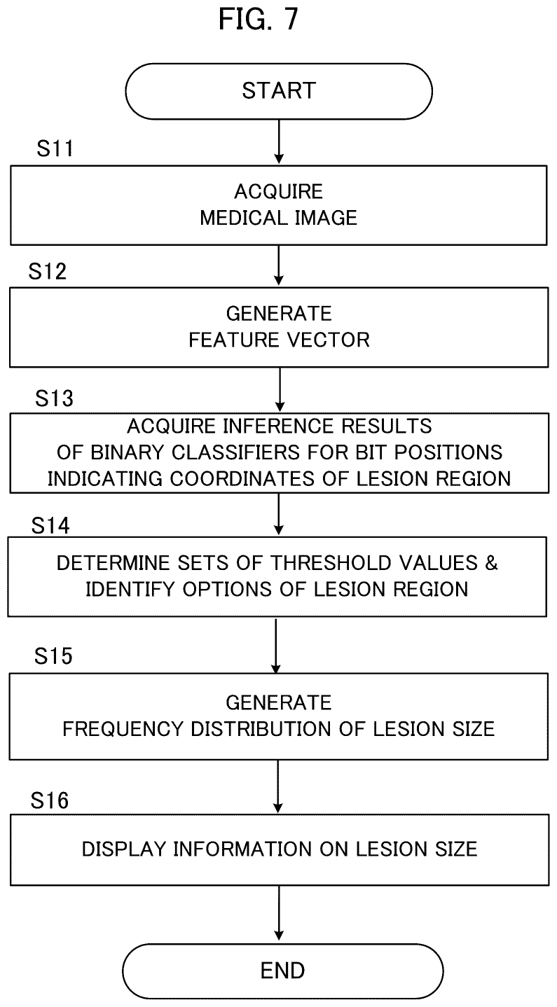

FIG. 7 is an example of a flowchart illustrating an outline of processing that is executed by the image processing device 1.

First, the image processing device 1 acquires a target medical image of evaluation (step S11). In this case, the image processing device 1 may receive, through the input device 4, a user input that specifies at least one of the examinee and/or the medical image.

Next, the image processing device 1 generates the feature vector of the medical image acquired at step S11 (step S12). In this instance, for example, the image processing device 1 acquires a feature vector output by the feature extractor by inputting the medical image into the feature extractor configured by referring to the feature extractor information D1.

Next, the image processing device 1 acquires the inference results of the binary classifiers for respective bit positions indicating the coordinates of the lesion region (step S13). In this case, for example, the image processing device 1 acquires N pieces of inference results for N bit positions output by the N binary classifiers by inputting the feature vector into each of the N binary classifiers configured by referring to the binary classifier information D2.

Then, the image processing device 1 determines threshold values to be compared with N inference results and identifies a plurality of options of the lesion region (step S14). In this case, the image processing device 1 identifies the values of respective bit positions for each set of the threshold value(s) through comparison between the inference results and the threshold value(s), and identifies the option of the lesion region for each set of the threshold value(s) based on the coordinates obtained by decoding the identified values. Thus, the image processing device 1 acquires M options of the lesion region, and identifies the lesion size for each of M options of the lesion region.

Then, the image processing device 1 generates a frequency distribution of the lesion sizes (step S15). In this case, the image processing device 1 generates the frequency distribution of lesion sizes of the M options of the lesion region. Thus, the image processing device 1 acquires, for example, the degree of confidence for each lesion size in accordance with the number of times in the corresponding bin.

Next, the image processing device 1 displays the information on the lesion size (step S16). In this case, for example, as shown in the first display example shown in FIG. 5 or the second display example shown in FIG. 6, the image processing device 1 displays information indicating a plurality of chances of the lesion size by the display device 3.

### (7) Modifications

Next, a description will be given of modifications of the first example embodiment described above. The following modifications may be applied to the first example embodiment described above in combination.

### (First Modification)

Instead of generating M options of the lesion region using N binary classifiers configured to infer the values for respective bit positions indicating the coordinates of the lesion region, the image processing device 1 may generate M options of the lesion region using M lesion detection models each configured to detect the lesion region.

In this case, for example, the image processing device 1 inputs the medical image to the M lesion detection model to acquire M inference results representing the lesion region from the M lesion detection models. Each lesion detection model is a machine learning model that has learned the relation between a medical image and a lesion region in the medical image, and is preliminarily trained through machine learning to take as input a medical image and output an inference result indicating a lesion region in the input medical image. For example, the lesion detection model may be a model (including a statistical model, hereinafter the same) that includes an architecture employed in any machine learning, such as a neural network and a support vector machine. Examples of the typical model which adopts such a neural network include Fully Convolutional Network, SegNet, U-Net, V-Net, Feature Pyramid Network, Mask R-CNN, and DeepLab. The lesion detection model is trained in advance on the basis of sets of an input image that conforms to the input format of the lesion detection model and correct answer data (e.g., a bounding box indicating a lesion region) which indicates the correct answer of the inference result that the lesion detection model should output upon taking the corresponding input image. In place of the feature extractor information D1 and the binary classifier information D2, the memory 12 stores the learned parameters of the M lesion detection models. The M detection models differ in at least one of architecture and/or the training data used for learning (i.e., sets of the input image and correct answer data described above). Therefore, the M lesion detection models output different inference results even when each lesion detection model takes the same medical image as input.

Even in this modification, the image processing device 1 can generate M options of the lesion region based on the inference results from the M lesion detection models and display information indicating a plurality of options of the lesion size.

### (Second Modification)

The image processing device 1 may display a plurality of options of the lesion size without generating the frequency distribution. In this case, for example, the image processing device 1 may display bounding boxes indicating M options of the lesion region on the target medical image of evaluation. In another example, the image processing device 1 may display bounding boxes indicating a plurality of options selected randomly or based on a predetermined rule from the M options of the lesion region on the target medical image of evaluation. Even in this case, the image processing device 1 can display information indicating a plurality of options of the lesion size.

### (Third Modification)

At least one of the feature extractor information D1, the binary classifier information D2, and/or the test information D3 may be stored in a storage device separated from the image processing device 1.

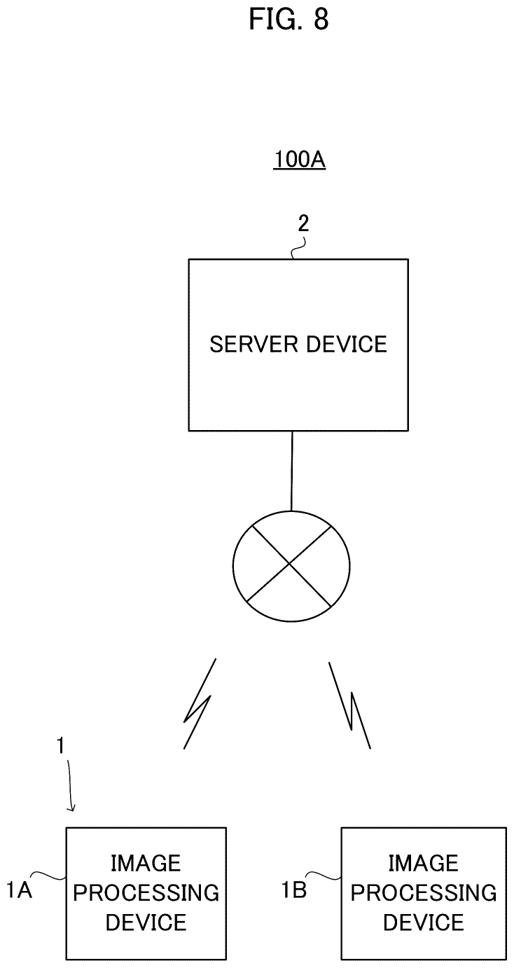

FIG. 8 is a schematic configuration diagram of a lesion evaluation system 100A. The lesion evaluation system 100A includes a server device 2 that stores at least one of the feature extractor information D1, the binary classifier information D2, and/or the examination information D3. In addition, the lesion evaluation system 100A includes a plurality of image processing devices 1 (1A, 1B, . . . ) which perform data communication with the server device 2 via a network.

In this instance, the image processing device 1 refers to at least one of the feature extractor information D1, the binary classifier information D2, and/or the examination information D3 via the network. In this case, the interface 13 of each image processing device 1 includes a communication interface such as a network adapter for performing data communication. In this configuration, each of the image processing devices 1 refers to the feature extractor information D1, the binary classifier information D2, and the examination information D3 in the same manner as in the above-described example embodiment, and can suitably perform the process relating to the evaluation of the lesion size. The server device 2 may execute at least a part of the process executed by the function blocks of the processor 11 of the image processing device 1 illustrated in FIG. 2 instead.

### Second Example Embodiment

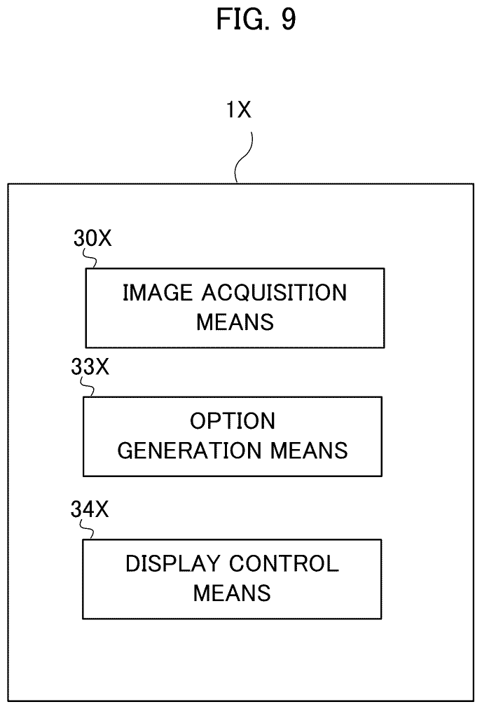

FIG. 9 is a block diagram of an image processing device 1X. The image processing device 1X includes an image acquisition means 30X, an option generation means 33X, and a display control means 34X. The image processing device 1X may be configured by a plurality of devices.

The image acquisition means 30X is configured to acquire a medical image. The image acquisition means 30X may acquire the medical image stored in the storage device, or may acquire the medical image directly from a medical device that generates the medical image. Examples of the image acquisition means 30X include the image acquisition unit 30.

The option generation means 33X is configured to generate, based on the medical image, plural options of a size of a region of interest included in the medical image. Examples of the option generation means 33X include the option generation/aggregation unit 33.

The display control means 34X is configured to cause a display device to display information on the plural options of the size of the region of interest. Examples of the display control unit 34X include the display control unit 34.

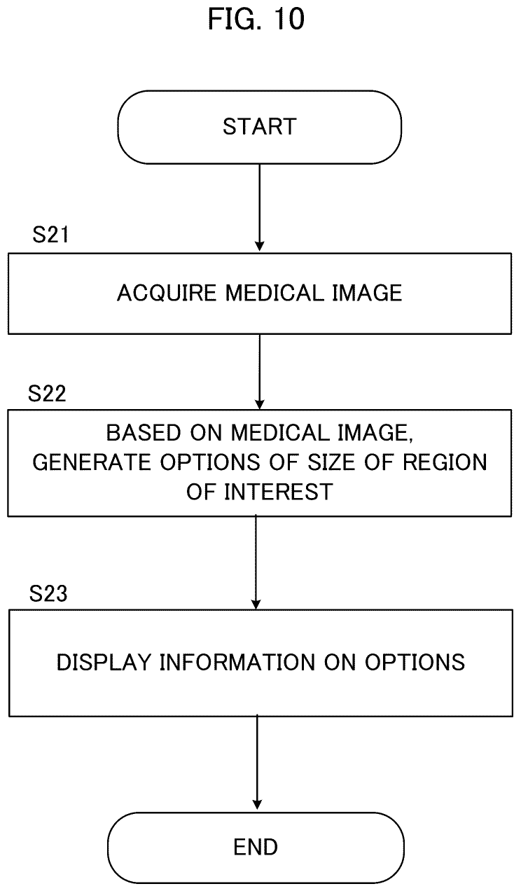

FIG. 10 is an exemplary flowchart illustrating the steps of the process that is executed by the image processing device 1X. The image acquisition means 30X acquires a medical image (step S21). The option generation means 33X generates, based on the medical image, plural options of a size of a region of interest included in the medical image (step S22). The display control means 34X causes a display device to display information on the plural options of the size of the region of interest (step S23).

According to the second example embodiment, the image processing device 1X can present a plurality of options of the size of the region of interest in the medical image to the user and suitably visualize a plurality of possible options regarding the size of the region of interest.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

In addition, some or all of the above-described example embodiments (including modifications, the same shall apply hereinafter) may also be described as follows, but are not limited to the following. Furthermore, within the range defined by the above-described example embodiments, regardless of the device, method, and storage medium described in the following Supplementary Notes, some or all of the configurations described in the following Supplementary Notes may be applied to any hardware, software, system and recording means (including the storage medium) for recording a software.

### [Supplementary Note 1]

An image processing device comprising:
an image acquisition means configured to acquire a medical image;
an option generation means configured to generate, based on the medical image, plural options of a size of a region of interest included in the medical image; and
a display control means configured to cause a display device to display information on the plural options.

### [Supplementary Note 2]

The image processing device according to Supplementary Note 1, further comprising
a frequency distribution generation means configured to generate a frequency distribution of the plural options of the size of the region of interest,
wherein the display control means is configured to cause the display device to display the information based on the frequency distribution.

### [Supplementary Note 3]

The image processing device according to Supplementary Note 2,
wherein the display control means is configured to cause the display device to display the information regarding respective degrees of confidence of the plural options based on the frequency distribution.

### [Supplementary Note 4]

The image processing device according to Supplementary Note 3,
wherein the display control means is configured to execute the instructions to cause the display device to display the plural options in association with the respective degrees of confidence.

### [Supplementary Note 5]

The image processing device according to Supplementary Note 1,
wherein the display control means is configured to cause the display device to highlight, on the displayed medical image, an option of the region of interest having a size selected from sizes of the plural options.

### [Supplementary Note 6]

The image processing device according to Supplementary Note 1,
wherein the display control means is configured to cause the display device to highlight, on the displayed medical image, an option of the region of interest having a largest size among the plural options.

### [Supplementary Note 7]

The image processing device according to Supplementary Note 1, further comprising
an inference means configured to make inference results for respective bit positions of coordinates of the region of interest in binary notation,
wherein the option generation means is configured to generate the plural options based on the inference results and plural sets of threshold value(s) to be compared with the inference results.

### [Supplementary Note 8]

The image processing device according to Supplementary Note 1, further comprising
a feature extraction means configured to extract features of the medical image, and
an inference means configured to make inference results for respective bit positions of coordinates of the region of interest in binary notation, based on the features and machine learning models for the respective bit positions,
wherein the machine learning models are trained, through machine learning, to learn a relation between features of an image and values at the respective bit positions of a region of interest in the image.

### [Supplementary Note 9]

An image processing method executed by a computer, comprising:
acquiring a medical image;
generating, based on the medical image, plural options of a size of a region of interest included in the medical image; and
causing a display device to display information on the plural options.

### [Supplementary Note 10]

A non-transitory computer readable storage medium storing a program executed by a computer, the program causing the computer to:
acquire a medical image;
generate, based on the medical image, plural options of a size of a region of interest included in the medical image; and
cause a display device to display information on the plural options.

### [Supplementary Note 11]

A storage medium storing a program according to Supplementary Note 10.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. Each example embodiment can be appropriately combined with other example embodiments. All Patent and Non-Patent Literatures mentioned in this specification are incorporated by reference in its entirety.

### DESCRIPTION OF REFERENCE NUMERALS

1, 1A, 1B, 1X Image processing device
2 Server device
3 Display device
4 Input device
11 Processor
12 Memory
13 Interface
100, 100A Lesion evaluation system

What is claimed is:

1. An image processing device comprising
at least one memory configured to store instructions, and
at least one processor configured to execute the instructions to:
acquire a medical image;
generate, based on the medical image, plural options of a size of a region of interest included in the medical image; and
cause a display device to display information on the plural options.

2. The image processing device according to claim 1,
wherein the at least one processor is configured to execute the instructions to
generate a frequency distribution of the plural options of the size of the region of interest, and
cause the display device to display the information based on the frequency distribution.

3. The image processing device according to claim 2,
wherein the at least one processor is configured to execute the instructions to cause the display device to display the information regarding respective degrees of confidence of the plural options based on the frequency distribution.

4. The image processing device according to claim 3,
wherein the at least one processor is configured to execute the instructions to cause the display device to display the plural options in association with the respective degrees of confidence.

5. The image processing device according to claim 1,
wherein the at least one processor is configured to execute the instructions to cause the display device to highlight, on the displayed medical image, an option of the region of interest having a size selected from sizes of the plural options.

6. The image processing device according to claim 1,
wherein the at least one processor is configured to execute the instructions to cause the display device to highlight, on the displayed medical image, an option of the region of interest having a largest size among the plural options.

7. The image processing device according to claim 1,
wherein the at least one processor is configured to execute the instructions to
make inference results for respective bit positions of coordinates of the region of interest in binary notation, and
generate the plural options based on the inference results and plural sets of threshold value(s) to be compared with the inference results.

8. The image processing device according to claim 1,
wherein the at least one processor is configured to execute the instructions to
extract features of the medical image, and
make inference results for respective bit positions of coordinates of the region of interest in binary notation, based on the features and machine learning models for the respective bit positions, and
wherein the machine learning models are trained, through machine learning, to learn a relation between features of an image and values at the respective bit positions of a region of interest in the image.

9. An image processing method executed by a computer, comprising:
acquiring a medical image;
generating, based on the medical image, plural options of a size of a region of interest included in the medical image; and
causing a display device to display information on the plural options.

10. A non-transitory computer readable storage medium storing a program executed by a computer, the program causing the computer to:
acquire a medical image;
generate, based on the medical image, plural options of a size of a region of interest included in the medical image; and
cause a display device to display information on the plural options.
